(19) United States
(12) Patent Application Publication
(10) Pub. No.: US 2024/0120073 A1
(43) Pub. Date: Apr. 11, 2024

(54) MEDICAL MANAGEMENT SYSTEM, MEDICAL MANAGEMENT DEVICE, AND MEDICAL MANAGEMENT METHOD

(71) Applicant: SONY GROUP CORPORATION, TOKYO (JP)

(72) Inventor: YUKI SUGIE, TOKYO (JP)

(21) Appl. No.: 18/546,201

(22) PCT Filed: Jan. 26, 2022
(86) PCT No.: PCT/JP2022/002754

§ 371 Date: Aug. 11, 2023

(30) Foreign Application Priority Data
Feb. 19, 2021 (JP) 2021-025095

(51) Int. Cl. G16H 30/40; G06F 3/14; G06T 5/50; G16H 30/20

(52) U.S. Cl. CPC G16H 30/40; G06F 3/14; G06T 5/50; G16H 30/20; G06T 2207/20221

(57) ABSTRACT

A medical management system according to an aspect of the present disclosure includes: an acquisition unit (31) that sequentially acquires image information for each patient; a priority setting unit (32) that dynamically sets a priority of processing on the image information for each patient; a processing unit (33) that determines a processing amount for each piece of the image information on the basis of the priority and performs processing on the image information for each patient on the basis of the determined processing amount for each piece of the image information; a generation unit (34) that generates integrated image information by integrating the image information for each patient on which the processing has been performed; and a display unit (42) that displays an integrated image on the basis of the integrated image information.

### FIELD

The present disclosure relates to a medical management system, a medical management device, and a medical management method.

### BACKGROUND

There has been proposed a surgery management system capable of remotely supporting an operator by providing a medical office strategy desk (central monitoring room) for monitoring individual patients in a plurality of operating rooms. This surgery management system is an example of a medical management system. In the surgery management system, it is assumed that information to be acquired is changed depending on use of an application such as image processing or the like on a server or a surgery status when monitoring or supporting a surgery. In order to cope with this, optimization of a processing load (server processing) of the server is demanded.

### CITATION LIST

### Patent Literature

Patent Literature 1: JP 2019-8766 A

### SUMMARY

### Technical Problem

Patent Literature 1 discloses optimization of server processing in an operating room, but as described above, optimization of server processing matching a medical office strategy desk capable of monitoring individual patients in a plurality of operating rooms is demanded.

Therefore, the present disclosure proposes a medical management system, a medical management device, and a medical management method capable of optimizing processing of handling image information for each patient.

### Solution to Problem

A medical management system according to the embodiment of the present disclosure includes: an acquisition unit configured to sequentially acquire image information for each patient; a priority setting unit configured to dynamically set a priority of processing for the image information for each patient; a processing unit configured to determine a processing amount for each piece of the image information on a basis of the priority and perform the processing on the image information for each patient on a basis of the processing amount determined for each piece of the image information; a generation unit configured to generate integrated image information by integrating image information for each patient for which the processing has been performed; and a display unit configured to display an integrated image on a basis of the integrated image information.

### BRIEF DESCRIPTION OF DRAWINGS

FIG. 1 is a diagram illustrating one example of a schematic configuration of a medical management system according to a first embodiment.

FIG. 2 is a diagram illustrating one example of a schematic configuration of a medical management device and a supervision room device according to the first embodiment.

FIG. 3 is a diagram illustrating one example of resource allocation processing of the medical management device according to the first embodiment.

FIG. 4 is a diagram illustrating one example of an integrated image according to the first embodiment.

FIG. 5 is a diagram illustrating one example of a schematic configuration of an operating room system according to the first embodiment.

FIG. 6 is a diagram illustrating one example of resource allocation processing of the medical management device according to a second embodiment.

FIG. 7 is a diagram illustrating one example of an integrated image according to a modification example.

FIG. 8 is a diagram illustrating a configuration example of hardware according to each embodiment or each modification example.

### DESCRIPTION OF EMBODIMENTS

Hereinafter, embodiments of the present disclosure will be described in detail with reference to the drawings. Note that a medical management system, a medical management device, and a medical management method according to the present disclosure are not limited by the embodiments. And, in each of the following embodiments, basically the same parts are denoted by the same reference signs, and redundant description is omitted.

One or more embodiments (including examples and modification examples) described below can each be implemented independently. Meanwhile, at least some of the plurality of embodiments described below may be combined with at least some of other embodiments as appropriate. The plurality of embodiments can include novel features different from each other. Therefore, the plurality of embodiments can contribute to solving different objects or problems, and can exhibit different effects.

The present disclosure will be described according to the following order of items.

1. Introduction

2. First Embodiment

2-1. One Example of Schematic Configuration of Medical Management System

2-2. Example of Schematic Configuration of Medical Management Device and Supervision Room Device

2-3. One Example of Resource Allocation Processing of Medical Management Device

2-4. One Example of Integrated Image

2-5. One Example of Schematic Configuration of Operating Room

2-6. Effects

3. Second Embodiment

3-1. One Example of Resource Allocation Processing of Medical Management Device

3-2. Effects

4. Other Embodiments

4-1. Modification Example 1

4-2. Modification Example 2

4-3. Other Modification Example

5. Hardware Configuration Example

6. Appendix

### 1. Introduction

Smart OR is gradually being developed along with IT of an operating room (OR). In surgery in which information of various modalities is integrated like Smart OR, there are many pieces of information to be confirmed, and it is expected that a surgical style in which not only the operator's decision but also decision making based on the advice of a skilled doctor (supervisor) at a remote medical office strategy desk is adopted. Meanwhile, in order to lower the price of the server, it is necessary to set a limit on the processing amount (e.g., videos of all operating rooms cannot be output at 4K), and thus, it is expected that there will be a limit on the image quality that can be displayed at the medical office strategy desk and the use of applications. For this reason, a system capable of displaying an appropriate image and activating an application at the medical office strategy desk is demanded. The same applies to monitoring in an intensive care unit (ICU). Therefore, by appropriately and dynamically optimizing server processing load allocation (resource distribution), processing for a request that changes from moment to moment is realized by a low cost server.

However, a low cost server may be not able to provide sufficient signal processing (e.g., 4K image processing, modality integration processing, annotation processing, and the like) for all video signals. This leads to missing a timing of issuing an instruction due to insufficient image quality or the like, and there is a risk of causing failure of surgery and a decrease in efficiency. Therefore, in order for the medical office strategy desk to issue an appropriate instruction instantaneously, it is necessary to execute appropriate signal processing on a video signal from an operating room at an appropriate timing.

For example, in principle, a processing amount of a video signal from each operating room is suppressed by reduction or frame thinning. When the processing amount is suppressed in this way, the image quality is deteriorated, and the information is insufficient to issue a sufficient instruction. Therefore, an operating room (patient) to be particularly presented to the supervisor is selected on the basis of a specific phase of an operating procedure, or an operating room voice and vital information, and the processing amount is not suppressed for the video of the operating room, and additional signal processing is performed in some cases, so that the supervisor is prevented from missing the instruction issuing timing due to image quality deterioration or the like.

That is, since the supervisor can issue an appropriate instruction at an appropriate timing by information of sufficient quality for issuing an instruction for which appropriate signal processing has been performed, it is possible to improve the operation efficiency and at the same time, reduce the fatigue of the supervisor. For example, the processing load is allocated to the operating room in which the supervision is considered to be necessary, and the processing load is not allocated to the operating room in which the supervision is considered to be unnecessary. Thus, since the total amount of calculation required for the server is suppressed, it is possible to realize processing for a request that changes from moment to moment by the low cost server. Details will be described in each embodiment.

### 2. First Embodiment

<2-1. One Example of Schematic Configuration of Medical Management System>

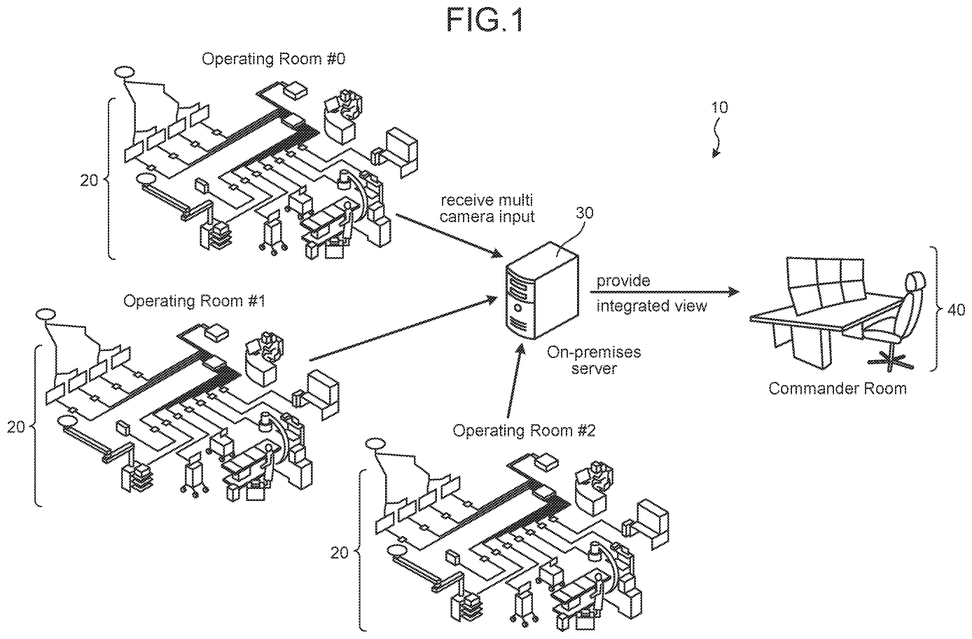

One example of a schematic configuration of a medical management system 10 according to the first embodiment will be described. FIG. 1 is a diagram illustrating one example of a schematic configuration of the medical management system 10 according to a first embodiment. In the example of FIG. 1, the medical management system 10 is a system that realizes that a supervisor gives an instruction to each operating room by voice, an annotation image, or the like while watching the states of a plurality of operating rooms.

As illustrated in FIG. 1, the medical management system 10 includes a plurality of operating room systems (operating room devices) 20, a medical management device 30, and a supervision room device 40. The operating room system 20, the medical management device 30, and the supervision room device 40 are configured to be able to transmit and receive various types of information. This transmission and reception are performed via a communication network such as wireless or wired.

The operating room system 20 is constructed for each operating room and includes various devices. In the example of FIG. 1, three operating room systems 20 are provided. Each of these operating room systems 20 acquires image information (video information) of a patient by various imaging devices (e.g., an endoscope, various cameras, an X-ray imaging device, and the like) installed in the operating room, and transmits the acquired image information of the patient to the medical management device 30. Note that the operating room system 20 will be described later in detail.

The medical management device 30 receives the image information for each patient transmitted from each operating room system 20, and executes resource allocation processing on the image information for each patient. Furthermore, the medical management device 30 executes various types of processing on the image information for each patient on the basis of resource allocation, integrates the processed image information to generate integrated image information, and transmits the integrated image information to the supervision room device 40. The medical management device 30 is one example of a server device and functions as a central intensive signal processing device. Note that the medical management device 30 will be described later in detail.

The supervision room device 40 receives the integrated image information transmitted from the medical management device 30, displays an integrated image G based on the received integrated image information, and provides the integrated image G to the supervisor. The supervision room device 40 is a device handled by a supervisor (remote monitoring staff), and is installed, for example, in a medical office strategy desk (central monitoring room) or the like. The supervisor visually recognizes the integrated image G displayed by the medical management device 30, performs input manipulation on the supervision room device 40, and issues an instruction to each operating room by voice, an annotation image, or the like. Examples of the supervisor include a skilled doctor of a remote medical office strategy desk, a specialized medical worker, and the like. Note that the supervision room device 40 will be described later in detail.

Herein, each operating room system 20, the medical management device 30, and the supervision room device 40 may be provided in a large hospital (e.g., a university hospital or the like). The medical management device 30 functions as, for example, a server device, but may be realized by cloud computing. Furthermore, each operating room system 20 may be provided in each of a plurality of hospitals, and the supervision room device 40 may be provided in a hospital different from the hospital in which the operating room systems 20 are provided. For example, the operating room system 20 may be provided in each hospital which is on a remote island or the like, and the supervision room device 40 may be provided in a university hospital which is in Tokyo or the like.

In addition to the operating room, the medical management system 10 can be applied to an intensive care unit (ICU), an advanced treatment unit (HCU), a circulatory disease intensive treatment unit (CCU), and the like. The operating room and the treatment room are examples of the medical room. Usually, there is one patient in the operating room, but there may be a plurality of patients in the treatment room other than a case where there is one patient. However, since there may be a plurality of patients in the operating room depending on the case, the number of patients in the operating room or the treatment room is not particularly limited.

<2-2. Example of Schematic Configuration of Medical Management Device and Supervision Room Device>

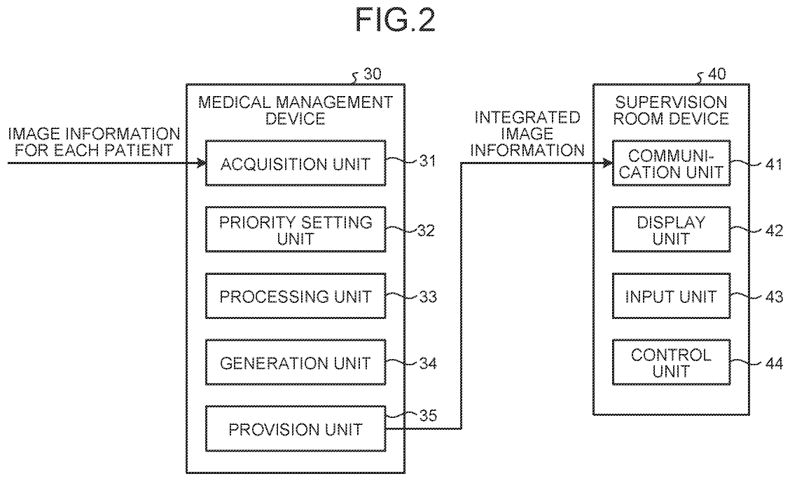

One example of a schematic configuration of the medical management device 30 and the supervision room device 40 according to the first embodiment will be described with reference to FIG. 2. FIG. 2 is a diagram illustrating one example of a schematic configuration of a medical management device 30 and a supervision room device 40 according to the first embodiment.

As illustrated in FIG. 2, the medical management device 30 includes an acquisition unit 31, a priority setting unit 32, a processing unit 33, a generation unit 34, and a provision unit 35.

The acquisition unit 31 sequentially receives and acquires the image information for each patient transmitted from each operating room system 20. The priority setting unit 32 dynamically (e.g., every time information is acquired,) sets the processing priority for the image information for each patient acquired by the acquisition unit 31 during service execution. The processing unit 33 determines a processing amount for each piece of image information on the basis of the priority set by the priority setting unit 32, and performs various types of processing on the image information for each patient on the basis of the determined processing amount for each piece of image information. Examples of the various types of processing include reduction processing, color conversion processing, CT superimposition processing, annotation processing, 4K image processing, and modality integration processing. The generation unit 34 integrates the processed image information for each patient to generate integrated image information. The provision unit 35 transmits the integrated image information generated by the generation unit 34 to the supervision room device 40.

The supervision room device 40 includes a communication unit 41, a display unit 42, an input unit 43, and a control unit 44.

The communication unit 41 transmits and receives various types of information to and from the medical management device 30 in a wired or wireless manner via a communication network. For example, the communication unit 41 receives various types of information such as integrated image information transmitted from the medical management device 30 and provides the information to the display unit 42. The display unit 42 displays various types of information (e.g., an integrated image G) such as the integrated image information provided from the communication unit 41. The input unit 43 accepts various manipulations such as an input manipulation from a supervisor who is a user. The control unit 44 issues an instruction to each unit such as the communication unit 41 and the display unit 42, and controls each unit.

Note that each functional unit such as the acquisition unit 31, the priority setting unit 32, the processing unit 33, the generation unit 34, the provision unit 35, the communication unit 41, the display unit 42, the input unit 43, or the control unit 44 described above may be configured by both or either one of hardware and software. The configuration of each functional unit is not particularly limited.

For example, each of the above-described functional units may be implemented by a computer including a central processing unit (CPU) or a micro control unit (MPU) executing a program stored in advance in a read only memory (ROM) using a random access memory (RAM) or the like as a work region. Furthermore, each functional unit may be implemented by, for example, an integrated circuit such as an application specific integrated circuit (ASIC) or a field-programmable gate array (FPGA).

Moreover, the display unit 42 may be implemented by, for example, a display device such as a liquid crystal display or an organic electro-luminescence (EL) display. Further, the input unit 43 may be implemented by, for example, a keyboard, a mouse, a touch panel, or the like. Note that the input unit 43 may be implemented by an input device that accepts an input manipulation by user's voice.

<2-3. One Example of Resource Allocation Processing of Medical Management Device>

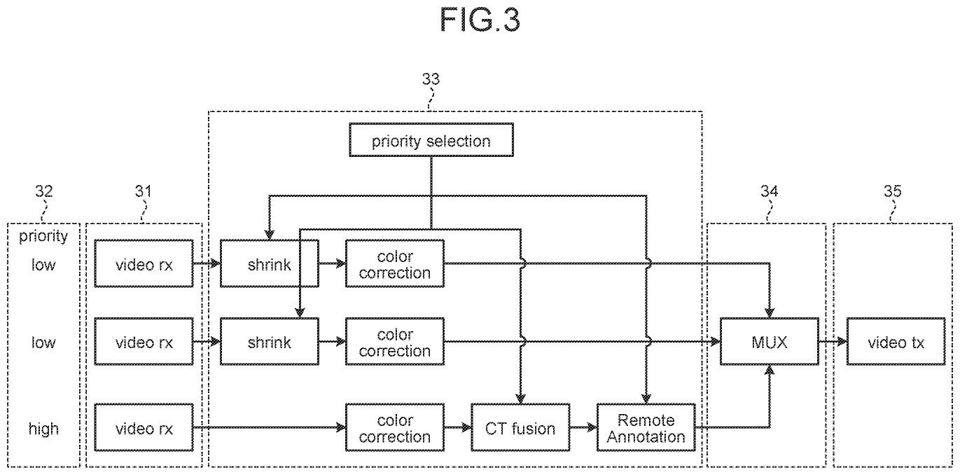

One example of resource allocation processing of the medical management device 30 according to the first embodiment will be described with reference to FIG. 3. FIG. 3 is a diagram illustrating one example of resource allocation processing of the medical management device 30 according to the first embodiment.

As illustrated in FIG. 3, the acquisition unit 31 receives and acquires a video signal (video information) of an individual patient from each operating room system 20 in real time (Video rx). The video signal is video information including a plurality of temporally continuous images. The video information is one example of the image information.

The priority setting unit 32 sets priority of the video signal for each patient. For example, on the basis of the patient selection information transmitted from the supervision room device 40, the priority setting unit 32 sets the priority of the video signal corresponding to the patient in the operating room to which the supervisor pays attention to high (high) and sets the priority of the video signal of other than the patient low (low).

For example, the supervisor manipulates the input unit 43 of the supervision room device 40 to select a patient (e.g., a patient in an operating room who needs to be thoroughly supervised) in the operating room of interest. In response to this selection, the control unit 44 generates patient selection information indicating the patient selected by the supervisor. The communication unit 41 transmits the patient selection information generated by the control unit 44 to the medical management device 30. As the input unit 43, for example, a touch panel is used. Note that, in a case where there is only one patient in the operating room, operating room selection information indicating the operating room may be used as the patient selection information.

The processing unit 33 allocates image size reduction processing (shrink) and color conversion processing (color correction) adapted to the display unit 42 of the supervision room device 40 to the video signal of which priority is set to low. On the basis of the processing allocation, the processing unit 33 performs shrink processing and color correction processing on the video signal whose priority is set to low. Note that the color correction processing is one example of a minimum process required for supervision by a supervisor.

Meanwhile, the processing unit 33 does not allocate the image size reduction processing (shrink) to the video signal of which the priority is set to high, but allocates the color conversion processing (color correction), the CT superimposition processing (CT fusion), and the remote annotation processing (remote annotation) for the display unit 42 of the supervision room device 40. On the basis of the processing allocation, the processing unit 33 performs color correction processing, CT superimposition processing, and remote annotation processing without shrinking image size on the video signal of which priority is set to high.

By dynamically controlling the contents of the signal processing in this manner, allocation of calculation resources is realized. In the example of FIG. 3, the priority setting unit 32 preferentially allocates a resource to a specific video signal. That is, the processing unit 33 determines the processing amount for each video signal on the basis of the priority, and performs processing on the video signal for each patient on the basis of the determined processing amount for each video signal.

The generation unit 34 integrates the video signals subjected to various types of processing by the processing unit 33 to generate integrated image information (MUX). The provision unit 35 transmits the integrated image information generated by the generation unit 34 to the supervision room device 40 (Video tx). The integrated image information is received by the supervision room device 40, and the integrated image G based on the integrated image information is displayed by the display unit 42 of the supervision room device 40.

According to the resource allocation processing as described above, the cost (price) of the entire system can be reduced by executing the signal processing on the medical management device 30 side instead of causing each camera of the operating room system 20 to perform the signal processing. Moreover, by including the priority setting unit 32, appropriate load distribution can be performed, and excessive configuration of the medical management device 30 can be avoided. Furthermore, the contents monitored and displayed by the supervision room device 40 can also distribute the result of processing by the medical management device 30 side. Since the video transmission band increases when signal processing is performed on the supervision room device 40 side, performing signal processing on the supervision room device 40 side does not match the infrastructure of the hospital.

<2-4. One Example of Integrated Image>

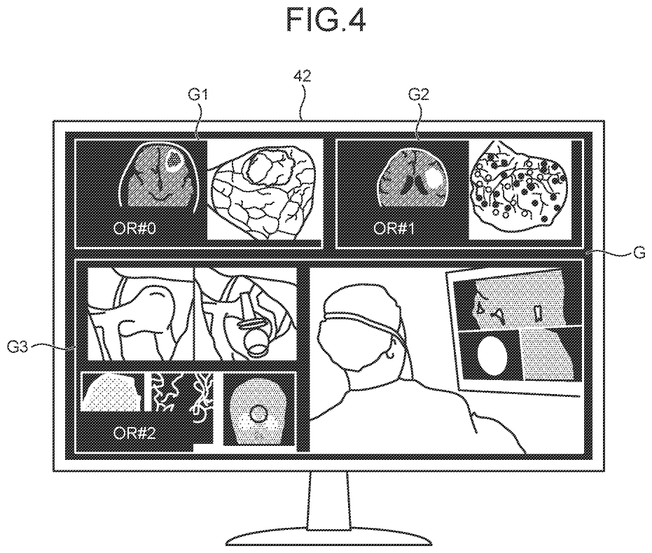

One example of the integrated image G according to the first embodiment will be described with reference to FIG. 4. FIG. 4 is a diagram illustrating one example of an integrated image G according to the first embodiment.

As illustrated in FIG. 4, the integrated image G includes an image G1 of a patient in an operating room (OR #0), an image G2 of a patient in an operating room (OR #1), and an image G3 of a patient in the operating room (OR #2). That is, the integrated image G is an image formed by integrating the images G1 to G3. The integrated image G is displayed by the display unit 42 of the supervision room device 40. In this manner, the display unit 42 presents the images (videos) of the plurality of cameras to the supervisor by picture in picture (PinP) or the like. Accordingly, the supervisor can visually recognize the integrated image G and give advice and instructions to a medical worker such as an operator and an assistant in each operating room.

Herein, for example, a user such as a supervisor (remote monitoring staff) performs input manipulation on the input unit 43 of the supervision room device 40, and gives advice or instructions to a medical worker such as an operator or an assistant in each operating room by voice, an annotation image, or the like. The advice, the instruction, and the like may be transmitted to each operating room system 20 via the communication network and the medical management device 30, or may be directly transmitted to each operating room system 20 via the communication network. Note that the advice or instruction by voice may be output to a medical worker such as an operator or an assistant in each operating room by a voice output device (e.g., a speaker) or the like of a facility such as a hospital.

<2-5. One Example of Schematic Configuration of Operating Room>

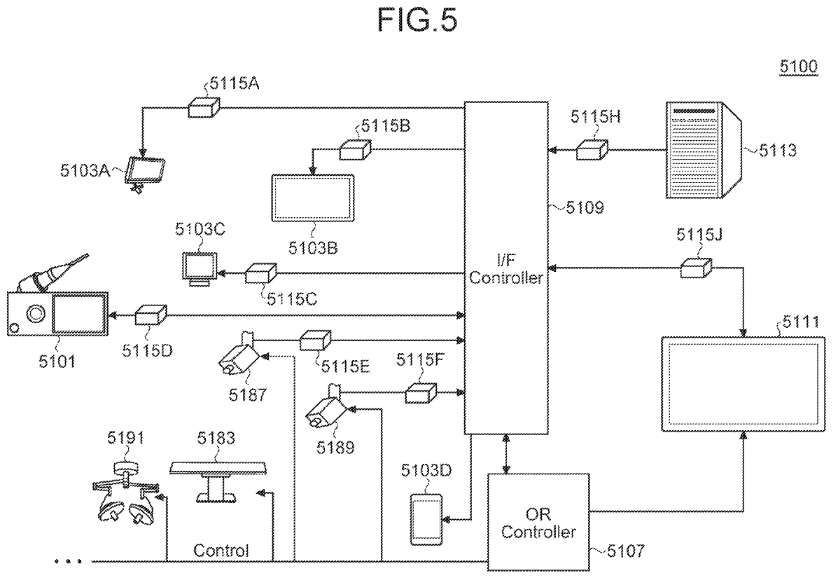

One example of a schematic configuration of an operating room system 5100 corresponding to the operating room system 20 according to the first embodiment will be described with reference to FIG. 5. FIG. 5 is a diagram illustrating one example of a schematic configuration of the operating room system 5100 according to the first embodiment. Note that, in the example of FIG. 5, an external server 5113 corresponds to a medical management device 30.

As illustrated in FIG. 5, the operating room system 5100 is configured by connecting a group of devices installed in an operating room so as to be capable of cooperating with one another via an operating room controller (OR Controller) 5107 and an input/output controller (IF Controller) 5109. The operating room system 5100 is configured using an Internet Protocol (IP) network capable of transmitting and receiving 4K/8K images, and transmits and receives input and output images and control information for the devices via the IP network.

Various devices can be installed in the operating room. FIG. 5 illustrates, as examples, a group of various devices 5101 for endoscopic surgery, a ceiling camera 5187 that is provided on the ceiling of the operating room and captures an area near the hands of an operator, an operating field camera 5189 that is provided on the ceiling of the operating room and captures an overall situation in the operating room, a plurality of display devices 5103A to 5103D, a patient bed 5183, and a light 5191. In addition to an endoscope illustrated in FIG. 5, various medical devices for acquiring images and videos, such as a master-slave endoscopic surgery robot and an X-ray imaging device, may be applied to the group of devices 5101.

The group of devices 5101, the ceiling camera 5187, the operating field camera 5189, and the display devices 5103A to 5103C are connected to the IF controller 5109 via IP converters 5115A to 5115F (hereinafter, denoted by reference numeral 5115 when not individually distinguished). The IP converters 5115D, 5115E, and 5115F on video source sides (camera sides) perform IP conversion on videos from individual medical image capturing devices (such as an endoscope, an operation microscope, an X-ray imaging device, an operating field camera, and a pathological image capturing device), and transmit the results on the network. The IP converters 5115A to 5115D on video output sides (monitor sides) convert the videos transmitted through the network into monitor-unique formats, and output the results. The IP converters on the video source sides function as encoders, and the IP converters on the video output sides function as decoders.

The IP converters 5115 may have various image processing functions, and may have functions of, for example, resolution conversion processing corresponding to output destinations, rotation correction and image stabilization of an endoscopic video, and object recognition processing. The image processing functions may also include partial processing such as feature information extraction for analysis on a server described later. These image processing functions may be specific to the connected medical image devices, or may be upgradable from outside. The IP converters on the display sides can perform processing such as synthesis of a plurality of videos (for example, picture-in-picture (PinP) processing) and superimposition of annotation information. The protocol conversion function of each of the IP converters is a function to convert a received signal into a converted signal conforming to a communication protocol allowing the signal to be transmitted on the network (such as the Internet). Any communication protocol may be set as the communication protocol. The signal received by the IP converter and convertible in terms of protocol is a digital signal, and is, for example, a video signal or a pixel signal. The IP converter may be incorporated in a video source side device or in a video output side device.

The group of devices 5101 belong to, for example, an endoscopic surgery system, and include, for example, the endoscope and a display device for displaying an image captured by the endoscope. The display devices 5103A to 5103D, the patient bed 5183, and the light 5191 are, for example, devices equipped in the operating room separately from the endoscopic surgery system. Each of these devices for surgical or diagnostic is also called a medical device. The OR controller 5107 and/or the IF controller 5109 controls operations of the medical devices in cooperation. When the endoscopic surgery robot (surgery master-slave) system and the medical image acquisition devices such as an X-ray imaging device are included in the operating room, those devices can also be connected as the group of devices 5101 in the same manner.

The OR controller 5107 controls processing related to image display in the medical devices in an integrated manner. Specifically, the group of devices 5101, the ceiling camera 5187, and the operating field camera 5189 among the devices included in the operating room system 5100 can each be a device having a function to transmit (hereinafter, also called a transmission source device) information to be displayed (hereinafter, also called display information) during the operation. The display devices 5103A to 5103D can each be a device to output the display information (hereinafter, also called an output destination device). The OR controller 5107 has a function to control operations of the transmission source devices and the output destination devices so as to acquire the display information from the transmission source devices and transmit the display information to the output destination devices to cause the output destination devices to display or record the display information. The display information refers to, for example, various images captured during the operation and various types of information on the operation (for example, body information and past examination results of a patient and information about a surgical procedure).

Specifically, information about an image of a surgical site in a body cavity of the patient captured by the endoscope can be transmitted as the display information from the group of devices 5101 to the OR controller 5107. Information about an image of the area near the hands of the operator captured by the ceiling camera 5187 can be transmitted as the display information from the ceiling camera 5187. Information about an image representing the overall situation in the operating room captured by the operating field camera 5189 can be transmitted as the display information from the operating field camera 5189. When another device having an imaging function is present in the operating room system 5100, the OR controller 5107 may also acquire information about an image captured by the other device as the display information from the other device.

The OR controller 5107 displays the acquired display information (that is, the images captured during the operation and the various types of information on the operation) on at least one of the display devices 5103A to 5103D serving as the output destination devices. In the illustrated example, the display device 5103A is a display device installed on the ceiling of the operating room, being hung therefrom; the display device 5103B is a display device installed on a wall surface of the operating room; the display device 5103C is a display device installed on a desk in the operating room; and the display device 5103D is a mobile device (such as a tablet personal computer (PC)) having a display function.

The IF controller 5109 controls input and output of the video signal from and to connected devices. For example, the IF controller 5109 controls input and output of the video signal based on controlling of the OR controller 5107. The IF controller 5109 includes, for example, an IP switcher, and controls high-speed transfer of the image (video) signal between devices disposed on the IP network.

The operating room system 5100 may include a device outside the operating room. The device outside the operating room can be a server connected to a network built in and outside a hospital, a PC used by a medical staff, or a projector installed in a meeting room of the hospital. When such an external device is present outside the hospital, the OR controller 5107 can also display the display information on a display device of another hospital via, for example, a teleconference system for telemedicine.

An external server 5113 is, for example, an in-hospital server or a cloud server outside the operating room, and may be used for, for example, image analysis and/or data analysis. In this case, the video information in the operating room may be transmitted to the external server 5113, and the server may generate additional information through big data analysis or recognition/analysis processing using artificial intelligence (AI) (machine learning), and feed the additional information back to the display devices in the operating room. At this time, an IP converter 5115H connected to the video devices in the operating room transmits data to the external server 5113, so that the video is analyzed. The transmitted data may be, for example, a video itself of the operation using the endoscope or other tools, metadata extracted from the video, and/or data indicating an operating status of the connected devices.

The operating room system 5100 is further provided with a central operation panel 5111. Through the central operation panel 5111, a user can give the OR controller 5107 an instruction about input/output control of the IF controller 5109 and an instruction about an operation of the connected devices. The user can switch image display through the central operation panel 5111. The central operation panel 5111 is configured by providing a touchscreen on a display surface of a display device. The central operation panel 5111 may be connected to the IF controller 5109 via an IP converter 5115J.

The IP network may be established using a wired network, or a part or the whole of the network may be established using a wireless network. For example, each of the IP converters on the video source sides may have a wireless communication function, and may transmit the received image to an output side IP converter via a wireless communication network, such as the fifth-generation mobile communication system (5G) or the sixth-generation mobile communication system (6G).

<2-6. Effects>

As described above, according to the medical management system 10 according to the first embodiment, the image information for each patient is sequentially acquired by the acquisition unit 31, and the priority of processing with respect to the acquired image information for each patient is dynamically set by the priority setting unit 32. The processing unit 33 determines the processing amount for each piece of image information on the basis of the set priority, and the processing unit 33 performs processing on the image information for each patient on the basis of the determined processing amount for each piece of image information. The image information for each patient subjected to the processing is integrated by the generation unit 34 to generate integrated image information, and the integrated image G is displayed by the display unit 42 on the basis of the generated integrated image information. In this manner, the priority of the image information for each patient is dynamically set, and the processing on the image information for each patient is performed on the basis of the processing amount for each piece of image information determined on the basis of the set priority, so that the processing of handling the image information for each patient can be optimized.

Furthermore, since the priority setting unit 32 can determine the processing amount for each piece of image information in accordance with the selection of the user by setting the priority on the basis of the selection of the user, it is possible to obtain the integrated image G reflecting the selection of the user.

Moreover, since the processing unit 33 can determine the processing amount for each piece of image information by changing the number of processing programs for at least one piece of image information among the pieces of image information for each patient on the basis of the priority, it is possible to easily optimize the processing of handling the image information for each patient. Note that examples of the processing program include programs (applications) such as shrinking processing, color correction processing, CT superimposition processing, and remote annotation processing.

### 3. Second Embodiment

<3-1. One Example of Resource Allocation Processing of Medical Management Device>

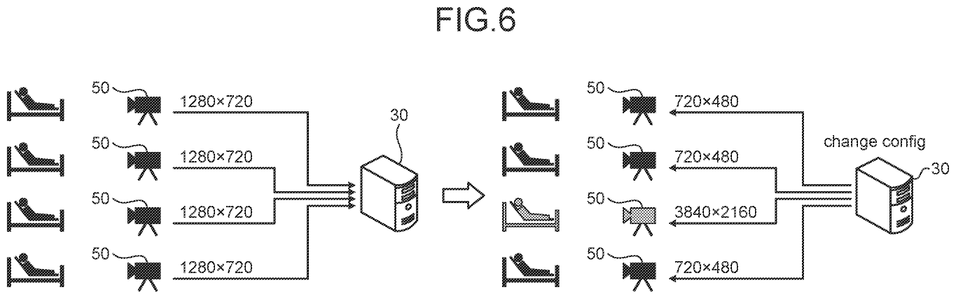

One example of resource allocation processing of the medical management device 30 according to a second embodiment will be described with reference to FIG. 6. FIG. 6 is a diagram illustrating one example of resource allocation processing of a medical management device 30 according to the second embodiment. Hereinafter, differences from the first embodiment will be mainly described, and other descriptions will be omitted.

Herein, for example, in an endoscope or the like in an operating room, it is difficult to assume that the number of pixels is changed due to influence on an operative procedure. However, for a camera in which only a supervisor (monitoring person) confirms video, such as an operative field camera in the operating room or a hospital bed camera in an ICU, the number of pixels may be controlled on a medical management device 30 side in accordance with patient selection information transmitted from a supervision room device 40 or a status of a patient (e.g., the phase of the surgery, the degree of bleeding, the facial expression of the patient, and the like). The priority setting unit 32 can decide the status for each patient on the basis of the image information for each patient. It is unnecessary that the operative field camera in the operating room and the camera in the treatment room always capture images with high pixels, and by capturing images with high pixels only in an important scene such as a bleeding scene, it is possible to further suppress the processing amount on the medical management device 30 side.

As illustrated in FIG. 6, a plurality of cameras 50 is provided in a treatment room (e.g., ICU, HCU, CCU, and the like). These cameras 50 are cameras capable of controlling the number of imaging pixels and the like from the medical management device 30. Note that, as the camera 50, for example, a camera capable of IP transmission may be used.

In a steady state, when detecting deterioration of the state of the patient in accordance with the status of the patient, for example, in a state where imaging is performed in HD 1280×720 p (see FIG. 6), the priority setting unit 32 sets the priority of the video signal of the patient to be higher than that of the video signal of another patient in order to grasp the state of the patient in more detail. The processing unit 33 changes the number of imaging pixels of the patient related to the video signal with the highest priority from 1280×720p to 3840×2160p (see FIG. 6) of 4K. At this time, the processing unit 33 changes the number of imaging pixels of a patient with low priority other than the patient from 1280×720p to 720×480p (see FIG. 6). By changing the image size of the camera and adjusting the data amount in this manner, it is possible to suppress the communication bandwidth of the video signal input to the medical management device 30 and suppress the calculation processing in the medical management device 30.

<3-2. Effects>

As described above, according to the second embodiment, the same effects as those of the first embodiment can be obtained. For example, since the processing unit 33 can determine the processing amount for each piece of image information by changing the data amount of at least one piece of image information among the pieces of image information for each patient on the basis of the priority (e.g., change from HD to 4K, change in bit depth or frame rate, and the like), the processing of handling the image information for each patient can be easily optimized.

<4. Other Embodiments>

The processing according to the previously mentioned embodiments may be performed in various different forms (modification examples) other than the above-described embodiments. For example, the system configuration is not limited to the above-described example, and may be various modes. This point will be described below. Note that, hereinafter, description of the same points as those of the medical management system 10 according to each embodiment will be omitted as appropriate.

<4-1. Modification Example 1>

In the first embodiment, the processing unit 33 performs resource allocation by changing the signal processing flow, that is, changes the number of processing programs (e.g., the number of applications) for the video signal (video information) on the basis of the priority (see FIG. 3), but resource allocation may be performed by the following control in addition to this.

For example, in a case where the processing unit 33 is a GPU, the resource allocation may be performed by controlling the number of instances (the number of processing programs) allocated to the video signal utilizing a multi instance Multi Instance Gpu (MIG) technology. The instance is an execution unit of the program. Furthermore, for example, the processing unit 33 may control the bit depth (e.g., 10 bit/pix→8 bit/pix) and the frame rate (e.g., 60 Hz→30 Hz) of the video signal and perform resource allocation. That is, the processing unit 33 may change the data amount of the video signal on the basis of the priority, or may change the communication bandwidth of the video signal on the basis of the priority. Even with such resource allocation, processing of handling image information for each patient can be optimized as in the first embodiment.

<4-2. Modification Example 2>

In each of the above embodiments, the priority setting unit 32 sets the priority of the video signal for each patient on the basis of the patient selection information transmitted from the supervision room device 40, but the present invention is not limited thereto. Specifically, the image of the patient in the operating room to which the supervisor pays attention is manually selected by an input unit 43 such as a touch panel, and the priority of the video signal of the patient in the operating room is set to be high. However, the priority setting is not limited thereto, and may be performed by any one of the following or a combination thereof.

The priority setting unit 32 analyzes the image information for each patient and automatically sets the priority on the basis of the analysis result. For example, the priority setting unit 32 may decide a status (e.g., the phase of the surgery, the degree of bleeding, the facial expression of the patient, and the like) for each patient on the basis of the image information for each patient, and perform the priority setting on the basis of the status. Furthermore, the priority setting unit 32 may set the priority on the basis of voice data (e.g., voice, volume, and the like for explicitly asking the supervisor for advice) from a medical worker such as a doctor or a nurse for a patient or on the basis of vital data (e.g., heart rate, blood pressure, oxygen saturation, and the like) for each patient. Furthermore, the priority setting unit 32 may perform the priority setting on the basis of the order data of the patient so as to periodically change the patient of interest. The order data is data indicating the order of patients and is set in advance, but can be changed by the user. For example, the priority is set to be highest every predetermined time (e.g., several minutes, several tens of minutes, or the like) in order from the top.

Usually, supervising many images (including moving images) increases fatigue of the supervisor, leading to erroneous determination. However, as described above, it is possible to reduce the burden on the supervisor by automating the selection of the image information of the patient in the operating room or the treatment room to be noted and performing the priority setting of the image information on the basis of the status of the patient in the operating room or the treatment room, the voice data of the medical worker, the vital data of the patient, the order data of the patient, and the like.

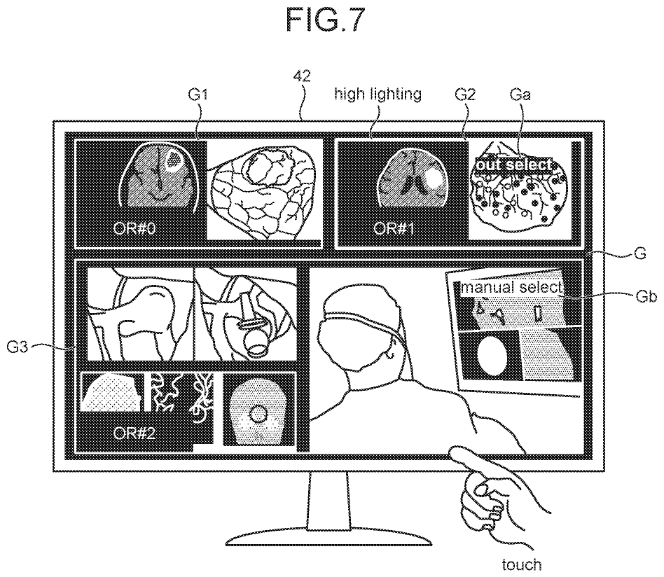

Herein, for example, in a case where the automatic priority setting based on the above analysis result and the manual priority setting based on the supervisor selection conflict with each other, the priority setting unit 32 preferentially executes the manual priority setting. In a case where the manual priority setting is preferentially executed, the priority setting unit 32 notifies of an image recommended to execute the automatic priority setting (change the priority) from among the integrated images G (the plurality of images G1 to G3). A candidate for which the execution of the automatic priority setting is recommended, for example, as illustrated in FIG. 7, the image G2 as the candidate may indicate to the supervisor that there is another candidate by a notification function such as blinking a PinP frame (high lighting). At this time, a word Ga “out select” may be superimposed on the image G2, and a word Gb “manual select” may be superimposed and displayed on the image G3.

Note that, as a notification function of emphasizing and notifying the candidate image G2, it is also possible to change the color of the frame or change the thickness of the frame, for example, in addition to the blinking of the frame. It is also possible to provide a sound output unit such as a speaker that outputs sound in addition to the display unit 42 and notify the supervisor that there are other candidates by sound such as voice.

In addition, in a case where the automatic priority setting based on the analysis result described above and the manual priority setting based on the selection by the supervisor conflict with each other, the priority setting unit 32 may preferentially execute the automatic priority setting. In this case, for example, the priority setting unit 32 can prioritize the automatic priority setting depending on whether the image information is the image information of the patient in the operating room or the image information of the patient in the treatment room. For example, the priority setting unit 32 may prioritize the manual priority setting in a case where the image information is image information of a patient in the operating room, and may prioritize the automatic priority setting in a case where the image information is image information of a patient in the treatment room.

<4-3. Other Modification Examples>

The processing according to each of the above-described embodiments and modification examples may be performed in various different forms (modification examples) in addition to the above-described embodiments and modification examples. For example, among the processes described in the above embodiments, all or part of the processes described as being performed automatically can be performed manually, or all or part of the processes described as being performed manually can be performed automatically by a known method. Moreover, the processing procedure, specific name, and information including various data and parameters illustrated in the document and the drawings can be arbitrarily changed unless otherwise specified. For example, the various types of information illustrated in each drawing are not limited to the illustrated information.

Furthermore, each component of each device illustrated in the drawings is functionally conceptual, and is not necessarily physically configured as illustrated in the drawings. That is, a specific form of distribution and integration of each device is not limited to the illustrated form, and all or a part thereof can be functionally or physically distributed and integrated in an arbitrary unit depending on various loads, usage conditions, and the like.

In addition, the above-described embodiments and modification examples can be combined as appropriate within a range in which the processing contents do not contradict each other. Furthermore, the effects described in the present specification are merely examples and are not limited, and other effects may be provided.

<5. Hardware Configuration Examples>

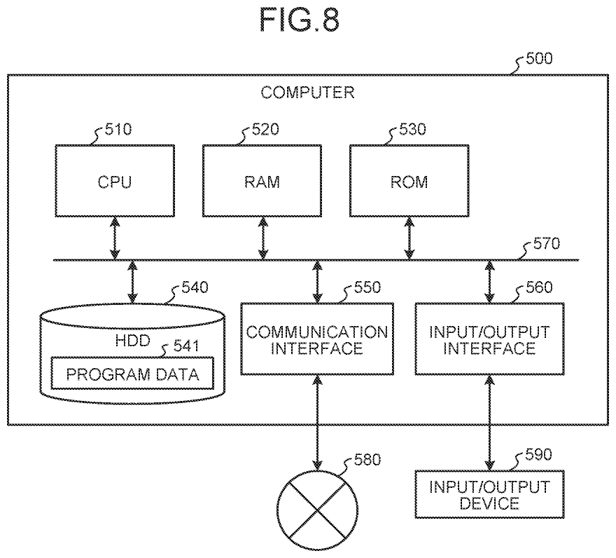

A specific hardware configuration example of the information apparatus such as a medical management device 30 or a supervision room device 40 according to each embodiment or each modification will be described. The information apparatus such as the medical management device 30 or the supervision room device 40 according to each embodiment or each modification may be realized by, for example, a computer 500 having a configuration as illustrated in FIG. 8. FIG. 8 is a diagram illustrating a configuration example of hardware that realizes functions of information apparatuses such as the medical management device 30 and the supervision room device 40 according to each embodiment or each modification example.

The computer 500 has a CPU 510, a RAM 520, a ROM 530, a hard disk drive (HDD) 540, a communication interface 550, and an input/output interface 560. Each unit of the computer 500 is connected by a bus 570.

The CPU 510 operates on the basis of a program stored in the ROM 530 or the HDD 540, and controls each unit. For example, the CPU 510 loads a program stored in the ROM 530 or the HDD 540 in the RAM 520, and executes processing for various programs.

The ROM 530 stores a boot program such as a basic input output system (BIOS) executed by the CPU 510 when the computer 500 is activated, a program depending on hardware of the computer 500, and the like.

The HDD 540 is a computer-readable recording medium that non-transiently records a program executed by the CPU 510, data used by the program, and the like. Specifically, the HDD 540 is a recording medium that records an information processing program according to the present disclosure as one example of the program data 541.

The communication interface 550 is an interface for connecting the computer 500 to an external network 580 (e.g., the Internet). For example, the CPU 510 receives data from another apparatus or transmits data generated by the CPU 510 to another apparatus via the communication interface 550.

The input/output interface 560 is an interface for connecting an input/output device 590 and the computer 500. For example, the CPU 510 receives data from an input device such as a keyboard and a mouse via the input/output interface 560. In addition, the CPU 510 transmits data to an output device such as a display, a speaker, or a printer via the input/output interface 560.

Note that the input/output interface 560 may function as a media interface that reads out a program or the like recorded in a predetermined recording medium (media). As the medium, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like is used.

Herein, for example, in a case where the computer 500 functions as the medical management device 30, the CPU 510 of the computer 500 executes the information processing program loaded on the RAM 520 to implement all or some of the functions of the acquisition unit 31, the priority setting unit 32, the processing unit 33, the generation unit 34, the provision unit 35, and the like. Moreover, the HDD 540 stores an information processing program and data (e.g., various images G1 to G3, an integrated image G, and the like) according to the present disclosure. Note that the CPU 510 reads and executes the program data 541 from the HDD 540, but as another example, these programs may be acquired from another device via the external network 580.

### 6. Appendix

Note that the present technology can also have the following configurations.

(1)

A medical management system comprising:
an acquisition unit configured to sequentially acquire image information for each patient;
a priority setting unit configured to dynamically set a priority of processing for the image information for each patient;
a processing unit configured to determine a processing amount for each piece of the image information on a basis of the priority and perform the processing on the image information for each patient on a basis of the processing amount determined for each piece of the image information;
a generation unit configured to generate integrated image information by integrating image information for each patient for which the processing has been performed; and
a display unit configured to display an integrated image on a basis of the integrated image information.

(2)

The medical management system according to (1), wherein

the priority setting unit sets the priority on a basis of user's selection.

(3)

The medical management system according to (1) or (2), wherein

the priority setting unit analyzes the image information for each patient and sets the priority on a basis of an analysis result.

(4)

The medical management system according to any one of (1) to (3), wherein

the priority setting unit determines a status for each patient on a basis of the image information for each patient and sets the priority on a basis of the status.

(5)

The medical management system according to any one of (1) to (4), wherein

the priority setting unit sets the priority on a basis of voice data of a medical worker for the patient.

(6)

The medical management system according to any one of (1) to (5), wherein

the priority setting unit sets the priority on a basis of vital data for each patient.

(7)

The medical management system according to any one of (1) to (6), wherein

the priority setting unit sets the priority on the basis of order data of the patient.

(8)

The medical management system according to any one of (1) to (7), wherein

the processing unit changes a number of processing programs for at least one piece of the image information of the image information for each patient on a basis of the priority.

(9)

The medical management system according to any one of (1) to (8), wherein

the processing unit changes a data amount of at least one piece of the image information of the image information for each patient on a basis of the priority.

(10)

The medical management system according to any one of (1) to (9), wherein

the processing unit changes a communication bandwidth of at least one piece of the image information of the image information for each patient on a basis of the priority.

(11)

The medical management system according to any one of (1) to (10), wherein

the priority setting unit preferentially executes manual priority setting in a case where executing manual priority setting in which the priority is set on a basis of selection of a user and automatic priority setting in which image information for each patient is analyzed and the priority is set on a basis of an analysis result.

(12)

The medical management system according to (11), wherein

the priority setting unit notifies an image recommended to execute the automatic priority setting from the integrated images in a case where the manual priority setting is preferentially executed.

(13)

A medical management device comprising:
an acquisition unit configured to sequentially acquire image information for each patient;
a priority setting unit configured to dynamically set a priority of processing for the image information for each patient;
a processing unit configured to determine a processing amount for each piece of the image information on a basis of the priority and perform the processing on the image information for each patient on a basis of the processing amount determined for each piece of the image information; and
a generation unit configured to generate integrated image information by integrating image information for each patient for which the processing has been performed.

(14)

A medical management method, by a computer, comprising:
sequentially acquiring image information for each patient;
dynamically setting a priority of processing for the image information for each patient;
determining a processing amount for each piece of the image information on a basis of the priority;
performing the processing on the image information for each patient on a basis of the processing amount determined for each piece of the image information; and
generating integrated image information by integrating the image information for each patient subjected to the processing.

(15)

A medical management device including a part of the medical management system according to any one of (1) to (12).

(16)

A medical management method using the medical management system according to any one of (1) to (12).

### REFERENCE SIGNS LIST

10 MEDICAL MANAGEMENT SYSTEM
20 OPERATING ROOM SYSTEM
30 MEDICAL MANAGEMENT DEVICE
31 ACQUISITION UNIT
32 PRIORITY SETTING UNIT
33 PROCESSING UNIT
34 GENERATION UNIT
35 PROVISION UNIT
40 SUPERVISION ROOM DEVICE
41 COMMUNICATION UNIT
42 DISPLAY UNIT
43 INPUT UNIT
44 CONTROL UNIT
50 CAMERA
G INTEGRATED IMAGE
G1 IMAGE
G2 IMAGE
G3 IMAGE

1. A medical management system comprising:
an acquisition unit configured to sequentially acquire image information for each patient;
a priority setting unit configured to dynamically set a priority of processing for the image information for each patient;
a processing unit configured to determine a processing amount for each piece of the image information on a basis of the priority and perform the processing on the image information for each patient on a basis of the processing amount determined for each piece of the image information;
a generation unit configured to generate integrated image information by integrating image information for each patient for which the processing has been performed; and
a display unit configured to display an integrated image on a basis of the integrated image information.

2. The medical management system according to claim 1, wherein
the priority setting unit sets the priority on a basis of user's selection.

3. The medical management system according to claim 1, wherein
the priority setting unit analyzes the image information for each patient and sets the priority on a basis of an analysis result.

4. The medical management system according to claim 1, wherein
the priority setting unit determines a status for each patient on a basis of the image information for each patient and sets the priority on a basis of the status.

5. The medical management system according to claim 1, wherein
the priority setting unit sets the priority on a basis of voice data of a medical worker for the patient.

6. The medical management system according to claim 1, wherein
the priority setting unit sets the priority on a basis of vital data for each patient.

7. The medical management system according to claim 1, wherein
the priority setting unit sets the priority on the basis of order data of the patient.

8. The medical management system according to claim 1, wherein
the processing unit changes a number of processing programs for at least one piece of the image information of the image information for each patient on a basis of the priority.

9. The medical management system according to claim 1, wherein
the processing unit changes a data amount of at least one piece of the image information of the image information for each patient on a basis of the priority.

10. The medical management system according to claim 1, wherein
the processing unit changes a communication bandwidth of at least one piece of the image information of the image information for each patient on a basis of the priority.

11. The medical management system according to claim 1, wherein
the priority setting unit preferentially executes manual priority setting in a case where executing manual priority setting in which the priority is set on a basis of selection of a user and automatic priority setting in which image information for each patient is analyzed and the priority is set on a basis of an analysis result.

12. The medical management system according to claim 11, wherein
the priority setting unit notifies an image recommended to execute the automatic priority setting from the integrated images in a case where the manual priority setting is preferentially executed.

13. A medical management device comprising:
an acquisition unit configured to sequentially acquire image information for each patient;
a priority setting unit configured to dynamically set a priority of processing for the image information for each patient;
a processing unit configured to determine a processing amount for each piece of the image information on a basis of the priority and perform the processing on the image information for each patient on a basis of the processing amount determined for each piece of the image information; and
a generation unit configured to generate integrated image information by integrating image information for each patient for which the processing has been performed.

14. A medical management method, by a computer, comprising:
sequentially acquiring image information for each patient;
dynamically setting a priority of processing for the image information for each patient;
determining a processing amount for each piece of the image information on a basis of the priority;
performing the processing on the image information for each patient on a basis of the processing amount determined for each piece of the image information; and
generating integrated image information by integrating the image information for each patient subjected to the processing.
